(19) United States
(12) Patent Application Publication
(10) Pub. No.: US 2019/0076185 A1
(43) Pub. Date: Mar. 14, 2019

(54) RECONFIGURABLE DESIGN FOR MULTIPLE AIRWAY TREATMENTS

(71) Applicant: AERIN MEDICAL, INC., Sunnyvale, CA (US)

(72) Inventors: Fred DINGER, Austin, TX (US); Andrew FRAZIER, Sunnyvale, CA (US)

(21) Appl. No.: 16/120,925

(22) Filed: Sep. 4, 2018

Related U.S. Application Data
(60) Provisional application No. 62/555,720, filed on Sep. 8, 2017.

(51) Int. Cl. A61B 18/14; A61B 18/02; A61B 17/3209; A61B 18/06

(52) U.S. Cl. CPC A61B 18/1485; A61B 18/02; A61B 2018/00327; A61B 18/06; A61B 17/3209

(57) ABSTRACT

Examples of devices, systems and methods for treating airway tissue disclosed herein include features that allow a device to change among multiple configurations. This configurability can allow the treatment of different tissue types and/or different locations within the airway. The different configurations may have different mechanical or energy delivery profiles suited for addressing different treatment goals, such as tissue shrinkage versus tissue shaping. Additionally, there may be sensors for determining a current configuration of a device and changing parameters to suit the current configuration.

### CROSS-REFERENCE TO RELATED APPLICATIONS

This application claims the benefit of U.S. Provisional Patent Application No. 62/555,720, filed Sep. 8, 2017, entitled, “RECONFIGURABLE DESIGN FOR MULTIPLE AIRWAY TREATMENTS.” The disclosure of this priority application is hereby incorporated by reference in its entirety into the present application.

### BACKGROUND

A number of different conditions of a patient's nasal airway may be addressed via intervention by a clinician. Successful intervention may necessitate the use of a variety of systems, devices, and methods to perform multiple different treatments. In addition, a clinician may begin a treatment believing that a particular kind of system, device, or method is needed and later realize a different kind would be more suitable.

Thus, a need exists to provide systems, devices, and methods to alleviate one or more drawbacks of traditional treatments, including long procedure times when multiple or differing treatments are required.

### SUMMARY

In general terms, this disclosure is relevant to devices, systems and methods for treating airway tissue. Disclosed devices and systems may include features that allow a device to have multiple configurations to provide greater flexibility during treatment. In some examples, the configurations are usable to treat different tissue types or different locations within the nasal airway. In addition, the different configurations may be suited for addressing different treatment goals, such as tissue shrinkage versus tissue shaping. The configurations may have different mechanical and/or energy delivery profiles. Additionally, the device may be able to sense the user reconfiguring the device and change other parameters to suit a different treatment automatically.

In one aspect of the present disclosure, a device for treating airway tissue may include: a handle; an outer shaft fixedly attached to the handle; an inner shaft disposed in the outer shaft; and a treatment element attached to a distal end of the inner shaft and configured to deliver energy to the airway tissue to modify at least one property of the tissue. The inner shaft is free to move in at least one direction within the outer shaft to change an orientation of the treatment element from a first configuration to a second configuration. In some embodiments, for example, the inner shaft is free to translate, relative to the outer shaft, and thus the first configuration is a retracted configuration, and the second configuration is an extended configuration. Alternatively or additionally, the inner shaft may be free to rotate in a plane perpendicular to a length of the inner shaft, relative to the outer shaft. Thus, the treatment element faces in a first direction in the first configuration and a second direction in the second configuration. Again, in some embodiments, the inner shaft is free to rotate and translate, relative to the outer shaft.

In some embodiments, the treatment element is adjustable relative to the inner shaft, to change an orientation of the treatment element. For example, the treatment element may be configured to rotate in a plane parallel to a length of the inner shaft. Optionally, the device may further include a position sensor for determining whether the treatment element is in the first configuration or the second configuration. In some embodiments, the device may be configured to operate using a first set of parameters when the treatment element is in the first configuration and a second set of treatment parameters when the treatment element is in the second configuration.

The outer shaft may include a lumen, and the inner shaft may be disposed in the lumen. The device may further include a fixation mechanism configured to lock the device in the first configuration or the second configuration. In some embodiments, the device is configured for treating nasal airway tissue, and the treatment element is sized to fit through a nostril of a nose.

In another aspect of the present disclosure, a method for treating an airway may involve: obtaining an airway treatment device; actuating a fixation mechanism of the treatment device; transitioning the device from a first configuration to a second configuration; positioning a treatment element within the airway proximate an airway tissue to be treated; and applying energy to the airway tissue with the treatment element. Using this method, the airway tissue at least partially maintains a modified property after the treatment element is removed and the airway tissue heals.

In one embodiment, actuating the fixation mechanism involves moving a peg out of a landing. In some embodiments, transitioning the device from the first configuration to the second configuration may involve moving an inner shaft of the treatment device relative to an outer shaft of the treatment device. For example, the inner shaft may be rotated and/or translated, relative to the outer shaft, according to various embodiments. In some embodiments, transitioning the device from the first configuration to the second configuration may involve adjusting a position of the treatment element relative to a shaft of the treatment device. For example, adjusting the position of the treatment element may involve rotating the treatment element in a plane parallel to a longitudinal axis of the shaft.

In some embodiments, the airway tissue treated with the method is nasal airway tissue. In such embodiments, positioning the treatment element may involve advancing the treatment element through a nostril.

In another aspect of the present disclosure, an airway treatment system may include a treatment device configured to deliver energy to tissue within an airway to modify at least one property of the tissue and a control system coupled to the treatment device. The control system may be configured to determine whether the treatment device is in a first configuration or a second configuration and modify one or more treatment parameters of the treatment device, based on whether the treatment device is in the first configuration or the second configuration.

In some embodiments, the treatment device further includes a supporting feature removably coupled to an attachment mechanism. The supporting feature may include, but is not limited to, a clamp portion, an electrode array, an incision forming device, a second treatment device, a positioning device, or a sensor array. In some embodiments, the treatment device may include a sensor configured to determine a configuration of the treatment device. The control system may then be configured to determine whether the treatment device is in the first configuration or the second configuration based on an output of the sensor.

In some embodiments, the device may include a fixation mechanism. In some embodiments, the device may include a treatment element having multiple selectable treatment portions. In such embodiments, the first configuration may involve an inner shaft of the plurality of selectable treatment portions being selected, and the second configuration may involve an outer shaft of the plurality of selectable treatment portions being selected.

These and other aspects and embodiments are described more fully in the detailed description below, in reference to the attached drawing figures.

### BRIEF DESCRIPTION OF THE DRAWINGS

FIG. 1 is a bottom view of a nose, looking upward into the nostrils;

FIG. 2 is a diagrammatic view of an airway treatment system, according to one embodiment;

FIG. 3 is a perspective view of a clamp airway treatment device, according to one embodiment;

FIG. 4 is a perspective view of an airway treatment device for creating an incision in surface tissue and treating a treatment site, according to one embodiment;

FIG. 5 is a block diagram depicting an array of electrodes of a treatment device arranged in a multi-channel configuration, according to one embodiment;

FIGS. 6A-6C are perspective views of an airway treatment device having an adjustable-height electrode array, according to one embodiment;

FIG. 7 is a block diagram, illustrating an example method for treating airway tissue, according to one embodiment;

FIGS. 8A and 8B are partial cutaway, side views of a multi-position airway treatment device having an adjustable length, according to one embodiment;

FIGS. 9A and 9B are partial cutaway, side views of a multi-position treatment device having a rotatable treatment element, according to one embodiment;

FIGS. 10A and 10B are front views of a treatment device with a rotatable treatment element that provides selective access to various treatment portions, according to one embodiment;

FIG. 11 is a partial cutaway, side view of a multi-position treatment device with a tiltable treatment element, according to one embodiment;

FIGS. 12A-12C are bottom views of a distal portion of a treatment device having a rotatable treatment element, according to one embodiment;

FIGS. 13A-13C are perspective views of a two-part treatment device formed from a supporting feature coupled to a base treatment device with an attachment element, according to one embodiment.

### DETAILED DESCRIPTION

Technologies disclosed in the present application are directed to devices, systems and methods for treating airway tissue. Examples of the technology include treatment devices and systems that are able to change among multiple different configurations. Such adjustable, configurable treatment devices and systems can facilitate treatment of different tissue types or different locations within a nasal airway. For example, the device may be able to transition from a first configuration suited for treating a nasal septum to a second configuration suited for treating nasal turbinates to a third configuration suited for treating lateral cartilage.

Different configurations may be suited to providing different kinds of treatment. For example, there may be configurations suited for providing treatment to previously treated and/or untreated tissue, and for shrinking and/or shaping tissue. For example, a first configuration of the device may be suited for providing treatment to untreated tissue, while a second configuration of the device may be suited for treating the already-treated tissue (e.g., shaping the shrunk tissue). For instance, a clinician may be able to shrink tissue while using the device in the first configuration, transition the device to the second configuration, and shape the shrunk tissue using the same (single) device. In another example, a first configuration of the device may be suited for treating a first kind of tissue (e.g., a first tissue type, such as cartilage or mucosa, or a first tissue location, such as turbinates or a septal tissue), and a second configuration of the device may be suited for treating a second kind of tissue. In another example, a first configuration is for providing a first part of a treatment and a second configuration is for providing a second part of the treatment.

Additionally, different configurations may provide treatment using different modalities. For example, a first configuration may provide treatment using radiofrequency energy, while a second configuration of the device may provide cryotherapy treatment. Similarly, different configurations may have different mechanical or energy delivery profiles. A first configuration may have a convex treatment surface profile, and a second configuration may have a concave treatment surface profile. A first configuration may have a high-energy treatment profile, and a second configuration may have a low-energy treatment profile. Additionally, the device may be able to sense the current configuration of the device and modify treatment parameters to suit the current configuration.

Various kinds of configurations are contemplated. In an example, a length of the treatment device can be modified. Modifying the length of a treatment device can facilitate treatment of different kinds of tissue and tissue in different locations within a patient's airway. For instance, in a first configuration, the treatment device may be relatively short and more suited for treating nasal tissue located nearer to the patient's nostrils. In a second configuration, the treatment device may be relatively longer and more suited for treating nasal tissue located deeper in the patient's nasal airway. During a procedure, a clinician may use the device in the first configuration to treat tissue in a first region of the patient's airway and then modify the device to assume the second configuration to treat tissue in a second region, deeper in the patient's nasal airway than the first.

In another example, a treatment element of the treatment device may be rotatable relative to a handle of the device. Rotation of the treatment element can allow the clinician to modify a treatment direction. For example, a clinician may prefer a particular treatment direction for treating particular anatomy or for a given way that the clinician holds the device. In some examples, the treatment element may have a variety of treatment portions, and rotating the treatment element may allow the clinician to select a specific treatment portion to use. For example, there may be a convex surface for shaping tissue on a first side of the treatment element and a concave surface for shaping tissue on a second side of the treatment element. The clinician may rotate the treatment element such that the proper side is exposed to a treatment site to allow for treatment. This adjustability provides for flexible treatment of different tissues/locations/etc. with a single treatment device.

In yet another example, a treatment element of a treatment device may be configured to be tilted in a plane substantially parallel to the length of the treatment device. This can facilitate treatment of different kinds of tissue. For example, a clinician may tilt the treatment element to facilitate treatment of a posterior aspect of a nasal turbinate. Tilting the treatment element can also facilitate transitioning the treatment device from a navigation configuration (e.g., a configuration that allows for easier navigation) to a treatment configuration (e.g., a configuration more suited for treatment of target tissue).

In a further example, a treatment element of the treatment device may be able to be rotated in a plane substantially perpendicular to a treatment direction (e.g., typically the treatment direction is perpendicular to a face of the treatment element).

In another example, there is a base treatment device having one or more attachment features suited for connecting a supporting feature to the base device to expand capabilities or functionality of the base device. For example, the supporting features may be clamps, sensors, treatment modalities and/or other components.

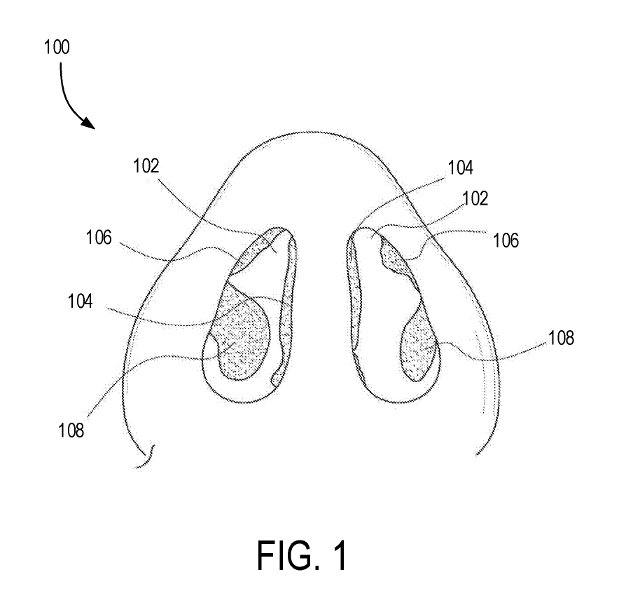

FIG. 1 illustrates a view of a nose as seen from the nostrils, including the nasal valve 102, between the nasal septum 104 and the upper lateral cartilage 106. The nasal turbinates 108 are also shown. Aspects of the technology disclosed herein can enable a treatment device to be customized to treat various aspects of an airway, and specifically a nasal airway in some embodiments, including the aspects shown and described in FIG. 1.

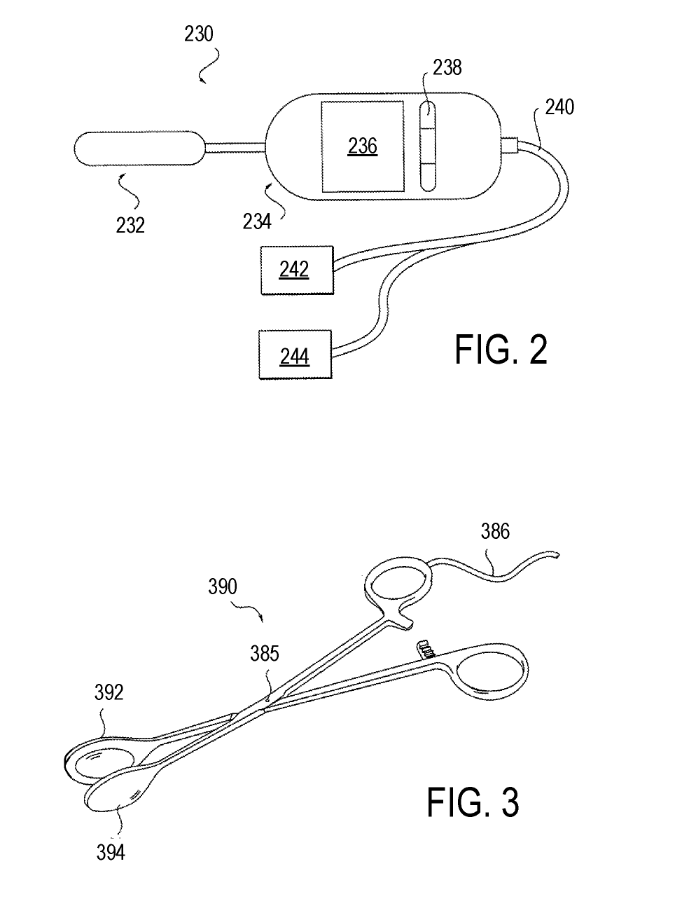

FIGS. 2-7 show and describe various systems, methods, and devices that can benefit from being adjustable (or “configurable”), according to aspects of the technology disclosed herein. In addition to the specific examples provided by those figures, aspects of the technology can be used with a variety of other kinds of treatment devices, including but not limited to those described in: U.S. Pat. No. 8,936,594, filed Jun. 13, 2012, entitled “METHODS AND DEVICES TO TREAT NASAL AIRWAYS”; U.S. Pat. No. 8,986,301, filed Sep. 13, 2013, entitled “METHODS AND DEVICES TO TREAT NASAL AIRWAYS”; U.S. Pat. No. 9,072,597, filed Jun. 30, 2014, entitled “METHODS AND DEVICES TO TREAT NASAL AIRWAYS”; and U.S. Pat. No. 9,415,194, filed Mar. 31, 2015, entitled “POST NASAL DRIP TREATMENT”, the entireties of which are incorporated herein by reference, for any and all purposes, including their disclosure of particular devices and systems for treating airways.

FIG. 2 illustrates an example treatment device 230. The device 230 includes a treatment element 232, which may be configured (e.g., sized or shaped) to be placed in, at, or near a subject's airway to deliver a desired treatment. In some embodiments, the device 230 may further include a handle 234, which may be sized and configured for easy handheld operation by a clinician. In some embodiments, the handle 234 may include a display 236 for displaying information to a clinician during treatment. Alternatively, the display 236 may be located on a “box” attached to the handle 234 by a cable 240 (box not shown in this embodiment).

In some embodiments, the information provided on the display 236 may include treatment delivery information (e.g., quantitative information describing the energy being delivered to the treatment element) and/or feedback information from sensors within the device and/or within the treatment element. In some embodiments, the display may provide information regarding treatment parameters, including time, power level, temperature, electric impedance, electric current, depth of treatment and/or other selectable parameters. In some embodiments, the display 236 may display information regarding a current configuration of the device 230.

In some embodiments, the handle 234 may also include one or more input controls 238 (e.g., buttons, dials, or joysticks). In some embodiments, controls may be incorporated into the display, such as by the use of a touch screen. In further embodiments, controls may be located on an auxiliary device, which may be configured to communicate with the treatment device 230 via analog or digital signals sent over a cable 240 or wirelessly, such as via BLUETOOTH, WIFI (or other 802.11 standard wireless protocol), infrared, or any other wired or wireless communication method. In some embodiments, the input controls 238 may be usable to modify a configuration of the device 230, for example the treatment element 232.

In some embodiments the device 230 may include an electronic control system 242 configured to control the timing, location, intensity and/or other properties and characteristics of energy or other treatment applied to targeted regions of an airway. In some embodiments, a control system 242 may be integrally incorporated into the handle 234. Alternatively, as illustrated, the control system 242 may be located in an external device, which may be configured to communicate with electronics within the handle 234. The control system 242 may include a closed-loop control system having any number of sensors, such as thermocouples, electric resistance or impedance sensors, ultrasound transducers, configuration sensors, or any other sensors configured to detect treatment variables or other control parameters.

The treatment system may also include a power supply 244. In some embodiments, the power supply 244 may be integrally incorporated within the handle 234. In alternative embodiments, a power supply 244 may be external to the handle 234. An external power supply 244 may be configured to deliver power to the handle 234 and/or the treatment element 232 by a cable or other suitable connection. The power supply 244 can be configured to provide power for an energy-based treatment from the treatment element 232. In some embodiments, a power supply 244 may include a battery or other electrical energy storage or energy generation device. In other embodiments, a power supply may be configured to draw electrical power from a standard wall outlet. In some embodiments, a power supply 244 may also include a system configured for driving a specific energy-delivery technology in the treatment element 232. For example, the power supply 244 may be configured to deliver a radio frequency alternating current signal to an RF energy-delivery element. Alternatively, the power supply may be configured to deliver a signal suitable for delivering ultrasound or microwave energy via suitable transducers. In further alternative embodiments, the power supply 244 may be configured to deliver a high-temperature or low-temperature fluid (e.g. air, water, steam, saline, or other gas or liquid) to the treatment element 232 by way of a fluid conduit.

In some embodiments, the treatment element 232 may have a substantially rigid or minimally elastic form that is sized and shaped such that it substantially conforms to a shape of a portion of the airway. In some embodiments, the treatment element 232 may have a curved shape, either concave or convex. In some embodiments, the shape of a fixed-shape treatment element 232 may be substantially in a shape to be imparted to target tissue (or “tissue to be treated”).

In some embodiments, the treatment element 232 and control system 242 may be configured to deliver treatment energy to create specific, localized tissue damage or ablation, which may stimulate the body's healing response to create desired conformational or structural changes in the tissue.

In some embodiments, the treatment element 232 and control system 242 may be configured to create specific localized tissue damage or ablation without the application of energy. For example the treatment element 232 may be configured to chemically cauterize or otherwise treat target tissue by delivering a chemical agent (e.g., a cauterizing agent, such as silver nitrate, trichloroacetic acid, or cantharidin) to the tissue. The treatment element 232 may include apertures configured to permit the cauterizing agent to pass through to the treatment site. In some embodiments, the treatment element 232 may aerosolize the cauterizing agent. Other delivery methods are also contemplated. The treatment element 232 may include a lumen through which the cauterizing agent passes. The lumen may be fluidly connected to a reservoir or container holding the cauterizing agent. The device may include an input control (e.g., a button or switch) configured to control delivery of the cauterizing agent. In some embodiments, the treatment element 232 includes an applicator that can be coated in a cauterizing agent (e.g., dipped in a reservoir of cauterizing agent, swabbed with cauterizing agent, etc.), and the coated treatment element applicator may be applied to the tissue to be treated. In some embodiments, the treatment element may be configured to apply cauterizing agent to the patient over a prolonged period of time (e.g., 30 seconds, 1 minute, 2 minutes, etc.). In some embodiments, the treatment element 232 includes shields configured to protect tissue surrounding the tissue to be treated from coming into contact with the cauterizing agent. In some embodiments, a separate element is used to shield tissue surrounding the tissue to be treated from coming into contact with the cauterizing agent. While such treatments may be performed without the application of energy, in some embodiments, they are performed in conjunction with energy treatments.

In some embodiments, a treatment element may be configured to treat tissue by applying treatment (e.g., energy, cryotherapy, or other treatments) from a position external to the patient's airway. For example, in some embodiments, the devices may be configured to apply energy from an element positioned outside a patient's body, such as on the skin.

In some embodiments, the device is configured to position the tissue to be modified. In some embodiments, the device includes features or mechanisms to pull, push or position airway tissue into a mold for re-shaping. For example, suction, counter traction, or compression between two parts of the device may be used.

In some embodiments, the treatment device includes one or more molds configured to re-shape tissue. The mold or re-shaping element may be fixed in size or may vary in size. The mold may also be fixed in shape or may vary in shape. For example, the size or shape of the element may be varied or adjusted to better conform to the airway of a patient. Adjustability may be accomplished using a variety of means, including, for example, mechanically moving the mold by way of joints, arms, guidewires, balloons, screws, stents, or scissoring arms. The mold may be adjusted manually or automatically.

In some embodiments, the mold or re-shaping element includes a separate or integrated energy-delivery or treatment element (e.g., an electrode). The treatment element may be fixed or adjustable in size. For example, the treatment element may be adjusted to better conform to a portion of the airway of a patient. In the case of a separate re-shaping element and treatment element, a distance between the two elements may either be fixed or adjustable. Adjustability may be accomplished using a variety of means, including, for example, mechanically moving the mold by way of joints, arms, guidewires, balloons, screws, stents, or scissoring arms, among other means.

In some embodiments, the mold or another part of the device is configured to deliver cooling (discussed in more detail below). In some embodiments, the mold or re-shaping element includes a balloon configured to reshape and/or deform tissue. A balloon may also be configured to deliver energy-based treatment using liquid or gas that is heated or cooled.

Various electrode arrangements may be used for applying energy to the tissue. These electrodes may, for example, deliver RF energy to preferentially shape the tissue to ameliorate symptoms, such as excessive airway resistance. In some embodiments, one or more electrodes may be used alone or in combination with a tissue shaping device or mold. In other embodiments, one or more electrodes may be integrally formed with a tissue shaping device or mold so that the electrodes themselves create the shape for the tissue. In some embodiments, the energy-delivery devices may use alternating current. In some embodiments, the energy-delivery devices may use direct current. In certain such embodiments, the energy-delivery device may include a configuration that uses a grounding pad.

In some embodiments, the term “electrode” refers to any conductive or semi-conductive element that may be used to treat the tissue. This includes, but is not limited to, metallic plates, needles, and various intermediate shapes such as dimpled plates, rods, domed plates, and other configurations. Electrodes may also be configured to provide tissue deformation in addition to energy-delivery. Unless specified otherwise, electrodes described can be monopolar (e.g., used in conjunction with a grounding pad) or bipolar (e.g., alternate polarities within the electrode body or used in conjunction with other tissue-applied electrodes).

In some embodiments, the treatment element 232 may include a substantially cylindrical central portion with an end-cap section that is semi-spherical, semi-ellipsoid, or another shape. The end-cap section can be located at proximal or distal ends of the treatment element 232. In alternative embodiments, the treatment element may include a substantially ellipsoid shape. In some embodiments, the treatment element 232 may include a clamp, such as to attach to a portion of a treatment site (e.g., as shown in FIG. 3).

FIG. 3 illustrates an example clamp treatment device 390. The treatment device 390 is structured as a clamp device configured to engage a targeted section of a treatment site with either a clamping force or a spreading force. In some embodiments, the treatment device 390 may include energy-delivery elements (of any type described herein) which may be powered by a fluid lumen or cable 386. The treatment device 390 includes a first clamp member 392 and a second clamp member 394 joined at a hinge point 385. The clamp members 392, 394 may be configured to attach to a treatment site. In some embodiments, the clamp members 392, 394 may be locked in a position relative to each other. In the illustrated embodiment, the first clamp member 392 includes an energy-based treatment element (e.g., applying energy to or removing energy from a treatment site). In some embodiments, the tissue-engaging tips may be removable to allow for sterilization and/or to allow for tips of a wide range of shapes and sizes to be used with a single clamp handle.

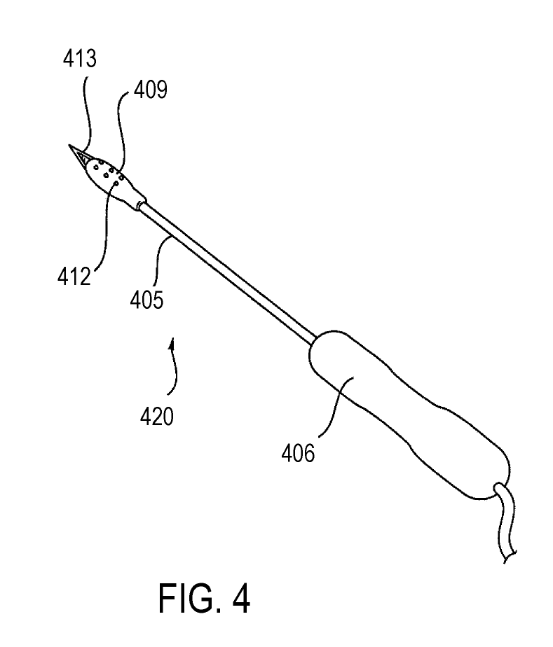

FIG. 4 illustrates an example treatment device 420 for creating an incision in surface tissue (e.g., airway mucosal tissue) and treating a treatment site. The device 420 may include a handle 406 having a shaft 405 extending therefrom. At an end of the shaft 405 opposite the handle 406, the device 420 may further include a head portion 409. The device 420 may include a blade 413 for creating an incision. In some embodiments, the blade 413 may be removable and/or retractable. The blade 413 may create an incision directly over a target tissue or may create an incision offset from the target tissue. When the incision is offset from the target tissue, the blade 413 may be used to dissect a path from the incision to the target tissue to contact the target tissue with an electrode array 412 of the head portion 409 of the device 420. When the electrode array 412 contacts the target tissue, the device 420 may be used to apply energy through the electrode array 412 to the target tissue.

In alternative embodiments, the blade 413 may be replaced by one or more other suitable cutting members, such as a cutting electrode that may cauterize tissue. As mentioned above, other incision-based treatment devices may include no cutting member, and whatever incision is used for a procedure may be formed with a separate cutting device.

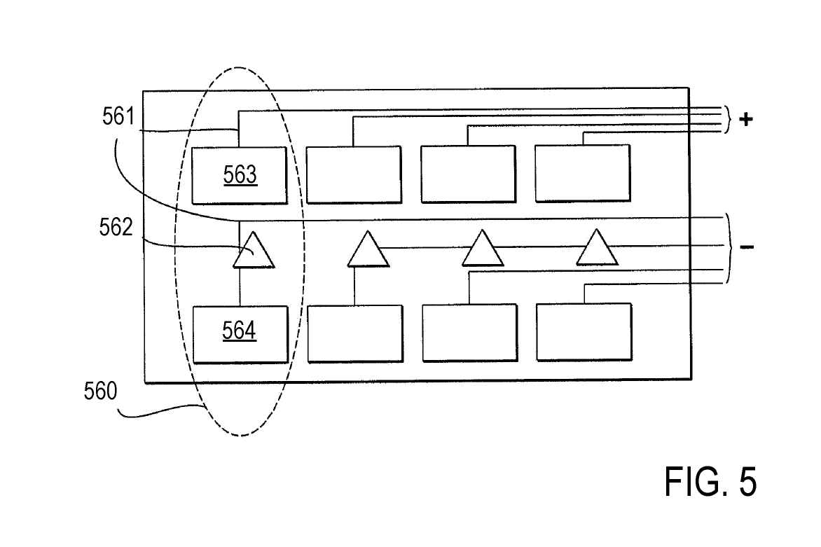

FIG. 5 is a block diagram depicting an array of electrodes of a treatment device arranged in a multi-channel configuration. Different treatment sites may have treatment surface areas of varying size and shape. Additionally, clinicians operating a treatment device may vary in skill, dexterity, and habits. Due to these variabilities, each electrode pair of the device may have varying degrees of contact with tissue of the treatment site. For a configuration in which all pairs of electrodes are controlled by one main electrical channel, this may lead to varying magnitudes of treatment energy passing through each electrode pair. The pair(s) of electrodes that have a higher degree of contact with the tissue may experience higher magnitudes of impedance in their individual circuit(s). Since treatment energy takes the path of least resistance, this may lead to treatment energy being diverted to pairs of electrodes that experience a relatively lower magnitude of impedance due to a relatively lower degree of tissue contact. Thus, it may be advantageous to control the treatment energy through each electrode to ensure repeatable treatments.

In some embodiments, each pair of electrodes may have a separate, controlled electrical channel to allow for different regions of the treatment element to be activated separately. In some embodiments, the separate activation of the pairs of electrodes may be based, in part, on the configuration of the treatment element or the device as a whole. In some embodiments, each electrode pair may be paired with its own thermocouple. By controlling the treatment energy flowing through each pair of electrodes using parameters including, but not limited to, temperature, a greater degree of control and accuracy over the treatment energy may be obtained, such that treatments may be repeatable.

As shown in FIG. 5, the treatment device may include one or more thermocouples 562 and an RF output channel 561 assigned to each electrode pair for feedback. An electrode pair may include a positive electrode 563 and a negative electrode 564. In some embodiments, the positive electrode 563 and the negative electrode 564 may be positioned opposite one another. Each electrode pair may have its own individual subsystem 560. The individual subsystem 560 may include a controlled RF output channel 561 and a thermocouple 562 to allow for independent adjustments. The thermocouple 562 may act as a feedback control to ensure that proper temperature is maintained at the treatment site.

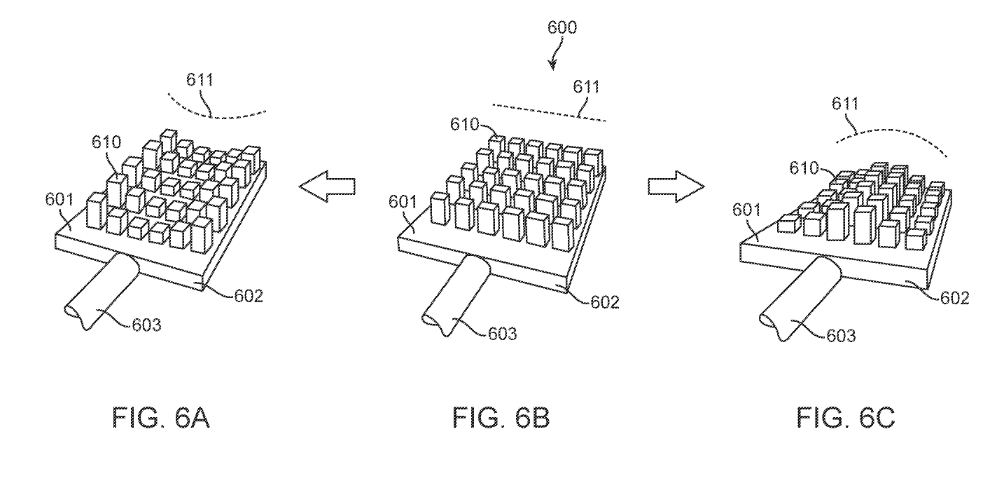

FIGS. 6A-6C illustrate an embodiment of a treatment device 600 including an array of electrodes 610 positioned on a surface of a treatment element 601. In some embodiments, as shown, the electrodes 610 may be arranged in a grid pattern. The electrodes 610 may be arranged in any pattern. One or more of the electrodes 610 may be extended or retracted to a preset height. It may be advantageous to manipulate the heights of the electrodes 610 to achieve a combination that forms a required treatment surface profile 611. The treatment surface profile 611 may include any combination of electrode numbers and heights. For example, FIG. 6A shows the electrodes 610 arranged in a concave configuration, FIG. 6B shows the electrodes 610 arranged in a flat configuration, and FIG. 6C shows the electrodes 610 arranged in a convex configuration. These are merely examples, however, and in alternative embodiments, the treatment element 601 may include any suitable number, shape, size and arrangement of electrodes 610, and the electrodes 610 may be arranged and adjusted into any suitable shape.

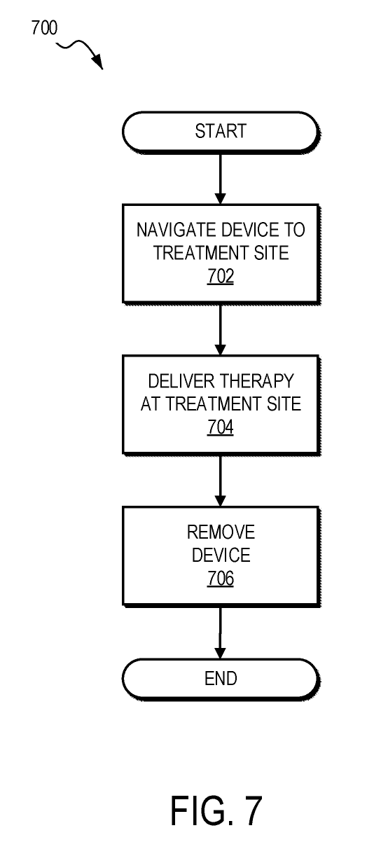

FIG. 7 illustrates a method 700 for treating airway tissue (including but not limited to upper airway or nasal tissue), according to one embodiment. In this embodiment, the method first involves the step 702 of navigating a treatment device to a treatment site. This may be accomplished, for example, by advancing a treatment element into the patient's nose through one of the nostrils. Alternatively, other methods for accessing the treatment site may be used. The next step 704 involves delivering therapy at the treatment site 704. The final step 706 may involve removing the device from the patient. Alternatively, the method 700 may involve repositioning the device and delivering therapy again. The repositioning and delivering steps may be performed as many times as desired to treat a given patient.

Typically, the method 700 may begin with an additional step of selecting a patient. For example, a clinician can select a patient having symptoms of an airway condition. In another example, the clinician can select a patient having or thought to be having an airway condition, such as excessive airway resistance, post-nasal drip, or a deviated septum.

The method 700 can further include preparing the device. Preparing the device may include removing the device from sterile packaging, assembling one or more components of the device, sterilizing the device, attaching the device to an energy source, and/or other preparatory work. In certain implementations, this step may include customizing the device to suit the particular needs of the patient and the clinician. This may include articulating, manipulating, or otherwise changing one or more components of the device. For example, a clinician may articulate or bend a shaft and/or the treatment portion to place the device in a first, desired configuration. The first, desired configuration may be selected to facilitate navigation of the anatomy of the particular patient to reach the desired treatment site.

In certain circumstances, preparing the device may follow a previous use or a previous attempted use of the treatment device to treat the same or a different treatment site. For example, the clinician may determine that the device is not suitable in its current state, remove the device, and then reconfigure the device in a more suitable format (e.g. a second configuration).

At step 702, the user navigates the treatment device to a treatment site. For example, the clinician may navigate the patient's anatomy with the device in the first configuration. The goal of the navigation may be to place the treatment element in contact with the treatment site. In an example, the treatment device is navigated through the nares to an internal nasal valve area of the nasal airway passage. In some embodiments, the clinician may pull the tip of the nose caudally and increase the diameter of the nares to facilitate access to the internal nasal valve for treatment. In some embodiments, access to the airway may be achieved endoscopically via the nares, or via the mouth and throat. In some embodiments, visualization devices may be incorporated or combined with treatment devices for navigation or treatment purposes.

In certain circumstances, advancing a portion of the device to a treatment site may follow the removal of the treatment device. In this circumstance, this step may include the clinician wholly or partially re-navigating the device to improve, for example, contact between the treatment portion and the treatment site.

During navigation, the clinician may perform one or more tests to determine whether proper contact with the treatment site has been made. For example, the clinician may activate one or more pairs of the electrodes. Based on measured results, the clinician may determine that proper contact has not been achieved, because an energy pathway could not be made between one or more pairs of electrodes and/or that one or more measured electrical parameters (e.g., impedance, voltage, current, etc.) is outside of a desired range. As another example, the clinician may attempt to apply pressure to the treatment site with the treatment portion and determine by feel whether proper contact has been made. As yet another example, the clinician may take a reading using a thermocouple to determine whether proper contact has been made.

Based on the one or more tests, the clinician may determine that proper contact has been made between the treatment portion and the treatment site. In this situation, the flow may move to the step 704, which involves delivering therapy to the treatment site. In certain circumstances, the clinician may determine that proper contact has not been made or that the device is otherwise unsuitable in its current state, and the clinician may reposition the device or modify a component thereof and re-navigate until proper contact is made.

At step 704, therapy is delivered at the treatment site. In this step, the clinician may cause the device to apply energy to or remove energy from the treatment site. For example, in certain implementations, a clinician may use the device to apply energy to mucosal tissue and/or an underlying tissue. In some examples, it may be desirable to press the treatment portion against airway tissue such that the tissue substantially conforms to the shape of the treatment element.

For instance, a concave shape may be formed or the tissue may be otherwise remodeled. In some examples, electrodes of the device may be non-penetrating (e.g., resist penetrating tissue, such as by having a blunt or rounded tip) electrodes that protrude from the treatment surface. The electrodes may create indentations in the tissue without piercing or otherwise penetrating the tissue. A portion of the tissue may enter and conform to the shape of a trough of the treatment device and contact a thermocouple. While the tissue is in this configuration, the clinician may activate one or more pairs of electrodes of the treatment device to deliver therapy to the treatment site. In certain implementations, delivering therapy to the treatment site may include delivering radio frequency energy from a first electrode on the treatment portion to a second electrode on the treatment portion to treat tissue such as mucosal tissue, cartilage, bone, muscle and/or skin, to modify a property of the tissue and thus treat a condition associated with the airway. The modification typically remains, in whole or in part, after the treatment element is removed and the tissue heals or otherwise recovers from the treatment.

At step 706, the device may be removed. In certain circumstances, the clinician may remove part or all of the device from the patient. The clinician may determine that one or more further adjustments may improve contact between the treatment portion and the treatment site or otherwise achieve improved therapeutic results. In such circumstances, the flow may move back to the start, and the clinician may re-prepare the device or the patient for treatment. For example, the clinician may articulate one or more components of the device to place the device in a second configuration. The clinician may then navigate to a new or the same treatment site and deliver therapy to the treatment site.

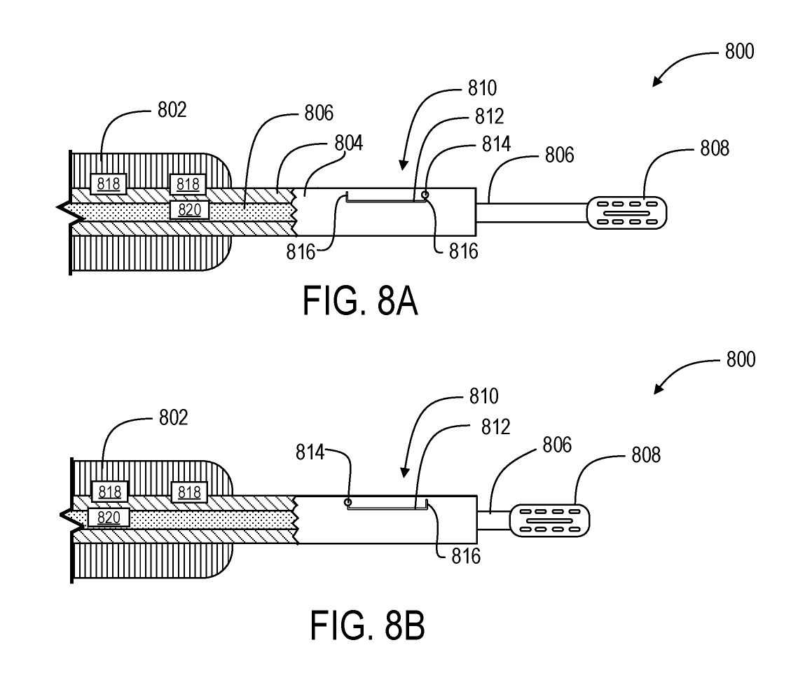

FIGS. 8A-13 illustrate various aspects of reconfigurable (or “adjustable”) technology, for methods, systems, and devices for treating airways.

FIGS. 8A and 8B illustrate side, partial cutaway views of an example multi-position treatment device 800 having an adjustable length. The multi-position treatment device 800 can include two or more related portions, at least one of which is movable relative to the other to customize the device 800. For example, extending from a handle 802 of the device 800 is an outer shaft 804. The outer shaft 804 can be fixed relative to the handle 802. An inner shaft 806 is slidably disposed relative the outer shaft 804. As illustrated in the cutaway portion of the example treatment device 800, the outer shaft 804 has a lumen in which the inner shaft 806 is slidably disposed. Extending from the end of the inner shaft 806 is a treatment element 808. In this manner, the outer shaft 804 and the inner shaft 806 form an adjustable shaft connecting the treatment element 808 to the handle 802. As illustrated, the treatment element 808 includes multiple pairs of bipolar electrodes and a thermocouple, though other configurations are also possible.

A fixation mechanism 810 can be used to control the movement of the inner shaft 806 relative to the outer shaft 804. The fixation mechanism 810 can take a variety of different forms including but not limited to a J-lock, a detent, a plug-in-channel, set screws, or other fixation mechanism. In some examples, the fixation mechanism 810 includes a groove, abutment, or other component that guides or limits movement of the inner shaft 806 relative to the outer shaft 804. The fixation mechanism 810 can be used, for example, to lock the outer shaft 804 and the inner shaft 806 into a particular relationship that defines multiple positions.

For example, FIG. 8A illustrates the inner shaft 806 being in an extended position relative the outer shaft 804, thereby defining an extended configuration. In the extended configuration the treatment element 808 is in a relatively distal position compared to other configurations. FIG. 8B illustrates the inner shaft 806 being in a retracted position relative the outer shaft 804, thereby defining a retracted configuration. In the retracted configuration, the treatment element 808 is in a relatively retracted position compared to other configurations.

In the example illustrated in FIGS. 8A and 8B, the fixation mechanism 810 includes a channel 812, in which a peg 814 is disposed. The peg 814 is coupled to the inner shaft 806, such that the peg 814 and the inner shaft 806 move together. In this configuration, the channel 812 guides and controls the movement of the peg 814 (e.g., preventing the peg 814 from leaving the confines of the channel 812), thereby also controlling the movement of the inner shaft 806. In the illustrated configuration, the channel 812 runs substantially parallel to the outer shaft 804. In this manner, the channel 812 cooperates with the peg 814 to allow motion of the inner shaft 806 in a direction substantially parallel to the inner shaft 806 and generally resists other motion of the inner shaft 806, such as twisting, while the inner shaft 806 is within a main portion of the channel 812. In some examples, the peg 814 is configured to be manipulated by the clinician to control the device (e.g., the peg 814 can extend to a particular height or otherwise be readily manipulated by the clinician). In other examples, the peg 814 and channel 812 need not be configured to be manipulated by the user. Instead, the user may use another component to manipulate the device, and one or more portions of the fixation mechanism 810 can be relatively inaccessible to the user (e.g., hidden within the housing of the device 800).

The channel 812 further includes landings 816, in which the peg 814 can rest. The landings 816 can be configured such that once the peg 814 is disposed in a landing 816, the landing 816 inhibits motion of the peg 814. In this manner, the landings 816 can be useful for maintaining a particular position of the peg 814 (and therefore also the inner shaft 806 and treatment element 808). In some examples, the fixation mechanism 810 can include a variety of different landings 816 to facilitate maintaining the treatment device 800 in various configurations. The landings 816 can take a variety of different configurations. In some examples, the landings 816 may be detents or catches in which the peg 814 are arrested. In the illustrated example, the landings 816 are offshoots from the channel 812 extending substantially perpendicular to the channel 812. A user may navigate the peg 814 into the landing 816 by twisting the outer shaft 804 and the inner shaft 806 relative to each other. In some examples, in order to modify engagement of the peg 814 and a landing 816, the user may need to push, pull, twist, or otherwise manipulate the peg 814 or another portion of the treatment device 800 (e.g., there may be a locking button on the handle 802). In some embodiments, the fixation mechanism 810 need not include landings 816. Instead, the fixation element 810 can include set screws, friction fits, or other ways of controlling the relative motion of the components.

The treatment device 800 can include a variety of sensors, electrical components, mechanical components, or other mechanisms for determining a configuration of the device 800. This can include, for example, detecting the position of the components of the treatment device 800, so that action can be taken in response thereto. For instance, as illustrated, there are a position sensor 818 and a position indicator 820 disposed within the treatment device 800. The position sensor 818 is a component for sensing the position of a component of the treatment device 800. The position indicator 820 is a component configured to indicate the position of a portion of the treatment device 800. In particular, the illustrated example shows the position indicator 820 as being disposed in relation to the inner shaft 806, such that the position indicator 820 moves with the inner shaft 806. Additionally, there are multiple position sensors 818 disposed within the handle 802 and the outer shaft 804 configured to sense the position of the position indicator 820. For example, a first position sensor 818 is disposed to detect when the inner shaft 806 is in a distal-most position (e.g., as shown in FIG. 8A). A second position sensor 818 is disposed to detect when the inner shaft 806 is disposed in a proximal position (e.g., as shown in FIG. 8B).

The position sensors 818 and the position indicator 820 can take a variety of different configurations. In some examples, the position indicator 820 is a conductive component that makes an electrical connection with the position sensor 818, which then produces a position output based on the connection. For instance, the position indicator 820 may be a conductive strip of material that completes a circuit for the position sensor 818. When the circuit is completed, a signal is sent from the position sensor 818, indicating that the position indicator 820 is in a position associated with the particular position sensor 818. In some examples, the position indicator 820 and the position sensor 818 can cooperate to form a potentiometer or a rheostat to provide a more continuous indication of position. In another example, the position indicator 820 may be a magnet, and the position sensor 818 may be a Hall effect sensor.

The output of the position sensor 818 can be provided to a component of the treatment device 800 or a system with which the treatment device 800 cooperates. For example, the position sensor 818 can provide an electrical or mechanical signal to a control system (e.g., control system 242) coupled to the treatment device 800. The control system can then modify operation of the treatment device 800 based on the signal.

For example, a first configuration of the device 800 may be a storage or safety configuration, and the control system may prevent use of the treatment device while in the first configuration. A second configuration of the device 800 may be an operational configuration that allows the clinician to operate the device.

In another example, the control system 242 may not allow operation of the device while the peg 814 is outside of a landing 816. Such a configuration may be determined if no sensor 818 detects the position indicator 820.

In yet another example, the control system 242 may cause the treatment device to operate according to a first set of treatment parameters while the position sensor 818 indicates a first output and may cause the treatment device to operate in a second set of treatment parameters while the position sensor 818 indicates a second output. Treatment parameters can include time, power level, temperature, electric impedance, electric current, and/or depth of treatment, among other selectable parameters. The treatment parameters may be selected based on a particular kind of tissue to be treated. For example, a first configuration may be a configuration for treating a particular region of the airway (e.g., a nasal septum, an inferior turbinate, a middle turbinate, a superior turbinate, a nasal valve region, Eustachian tube opening, mouth, throat, etc.) or a particular area of tissue (e.g., mucosal tissue, submucosal tissue, skin, upper lateral cartilage, lower lateral cartilage, nerve tissue, muscle tissue, cartilage, bone, etc.). The second configuration may be a configuration for treating another region of the airway and/or another kind of tissue.

In a further example, the parameters may be customized for a particular configuration. For instance, two or more clinicians may use a same treatment device, each having different preferences for use. A first configuration may select first parameters for a first clinician, and a second configuration may select second parameters for a second clinician.

In order to move the treatment device 800 from a first configuration to a second configuration, a user may apply force to disengage or otherwise modify a portion of the fixation mechanism 810 and then move one or more components of the treatment device 800. For example, to move from the configuration shown in FIG. 8A to the configuration shown in FIG. 8B, a user may begin by rotating the inner shaft 806 relative to the outer shaft 804, to move the peg 814 out of the landing 816 and into a main portion of the channel 812. This allows motion of the inner shaft 806 (and thus the treatment element 808) in a manner allowed by the channel 812, including movement of the treatment element 808 toward the handle, thereby decreasing an overall length of the treatment device 800. Once the treatment element 808 is in a desired position relative to the rest of the device 800, the user may activate the fixation mechanism 810. In particular, the user may twist the inner shaft 806 relative to the outer shaft 804 to place the peg 814 in a proximal landing 816 to arrive at the configuration illustrated in FIG. 8B.

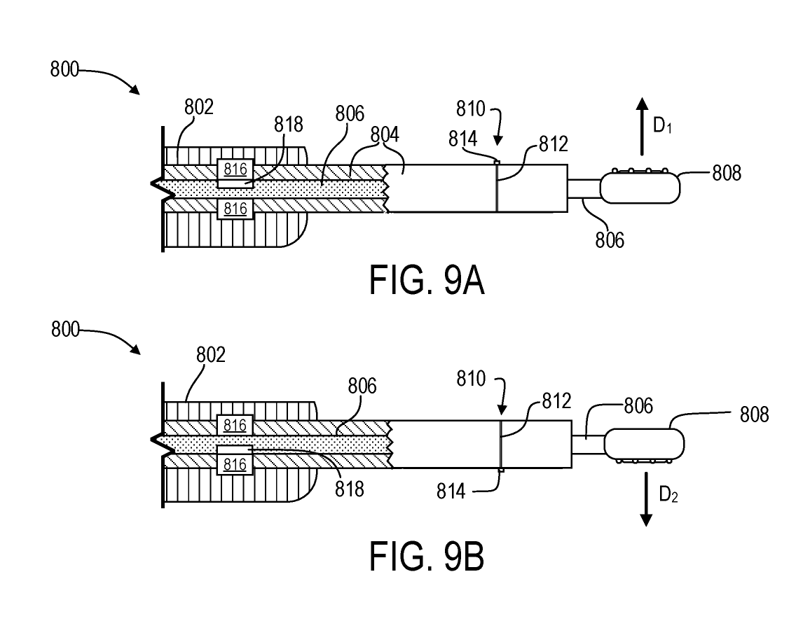

FIGS. 9A and 9B illustrate an example of a multi-position treatment device 900 having an adjustable treatment direction D₁, D₂. In particular, treatment device 900 is configured for rotation of the treatment element 908 in a plane substantially perpendicular to a length of the treatment device 900. FIG. 9A illustrates the treatment device 900 in a first configuration. In the first configuration, the treatment element 908 is oriented relative to the handle 902 to allow for treatment in direction D₁. FIG. 9B illustrates an example second configuration, in which the treatment element 908 is oriented relative to the handle 902 to allow for treatment in direction D₂. Direction D₂ is rotated approximately 180 degrees relative to direction D₁ in a plane substantially perpendicular to the length of the treatment device 900.

The arrangement of the outer shaft 904 and the inner shaft 906 can be configured to allow for rotation of components of the treatment device 900 in a plane substantially perpendicular to the length of the treatment device 900. For example, as shown, the outer shaft 904 is a tube in which the inner shaft 906 is disposed and can rotate.

The fixation mechanism 910 is configured to allow for the rotation. For example, the illustrated example includes a channel 912 oriented substantially circumferentially around the outer shaft 904, to allow for rotation of the peg 914 (and thus rotation of the second element 906 and the treatment element 908) around the circumference of the outer shaft 904. The fixation mechanism 910 can be configured in a variety of ways to inhibit unwanted motion of portions of the device 900. As with the example of FIGS. 8A and 8B, there may be landings to inhibit unwanted motion of components. In other examples, there may be other kinds of mechanisms.

The position sensor(s) 918 and the position indicator 920 can be arranged to detect rotation. For example, one position sensor 918 can be disposed circumferentially around the outer shaft 904, and the position indicator 920 can be disposed on a relatively small section of the inner shaft 906. Based on which portion of the position sensor 918 detects the position indicator 920, the position sensor 918 can provide an output. In another example, there may be multiple position sensors 918, each configured to detect a rotated position of the position indicator 920.

Rotating the treatment element 908 need not be just for changing a treatment direction. In some examples, the treatment element 908 may be rotated to access different features of the treatment element relative to the treatment direction.

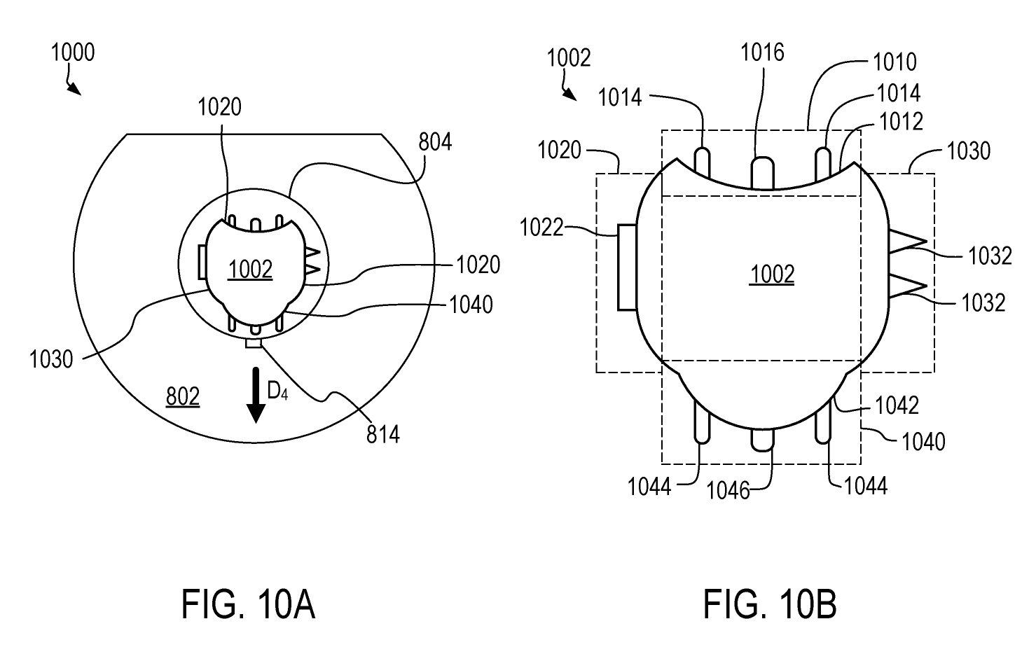

FIGS. 10A and 10B illustrate an example of a treatment device 1000 with a rotatable treatment element 1002 that provides selective access to various treatment portions 1010, 1020, 1030, 1040.

FIG. 10A illustrates an end-on view of the treatment device 1000. The device 1000 can be arranged to provide treatment in a particular direction D₄. Direction D₄ may vary, based on a variety of parameters, including clinician preference. For example, the treatment direction D₄ as shown in FIG. 10A is the same regardless of which treatment portion 1010, 1020, 1030, 1040 is selected. As illustrated in FIG. 10B, the treatment element 1002 has a variety of treatment portions 1010, 1020, 1030, 1040 arrayed circumferentially around the treatment element 1002. Each of the portions 1010, 1020, 1030, 1040 is adapted to provide a particular kind of treatment. The device 1000 is customizable by rotating the treatment element 1002 (e.g., as shown or described in relation to FIGS. 9A and 9B) to allow for treatment in the treatment direction D₄ by a variety of the treatment portions. For example, in the configuration shown in FIG. 10A, the device 1000 is arranged such that providing treatment in direction D₄ involves providing treatment with treatment portion 1040. However, the clinician may cause the treatment element 1002 to be rotated, such that a different treatment surface is arranged to provide treatment and direction D₄. Thus, the device 1000 provides multiple different types of treatment surfaces the clinician can use in a given procedure.

FIG. 10B illustrates a detailed view of the treatment element 1002 and its various treatment portions 1010, 1020, 1030, 1040. For example, in a first treatment portion 1010, there are one or more pairs of non-penetrating (e.g., blunted) bipolar electrodes 1014 disposed on a concave treatment surface 1012. The treatment portion 1010 may further include a thermocouple or other sensor 1016 disposed between the pairs of electrodes 1014. A second treatment portion 1020 includes a treatment mechanism 1022 configured to provide a treatment different from the treatment provided by portion 1010. For example, where treatment portion 1010 provides treatment using bipolar electrodes 1014, treatment mechanism 1022 can be configured to provide cryotherapy, chemical therapy, or another kind of treatment. Treatment portion 1030 includes at least one pair of bipolar penetrating (e.g., needle) pairs of electrodes 1032. Treatment portion 1040 includes one or more pairs of non-penetrating bipolar electrode pairs 1044 and a thermocouple or other sensor 1046 disposed on a convex treatment surface 1042. A clinician may access these various treatment regions by rotating the treatment element 1002 into a desired rotation relative to the rest of the device 1000.

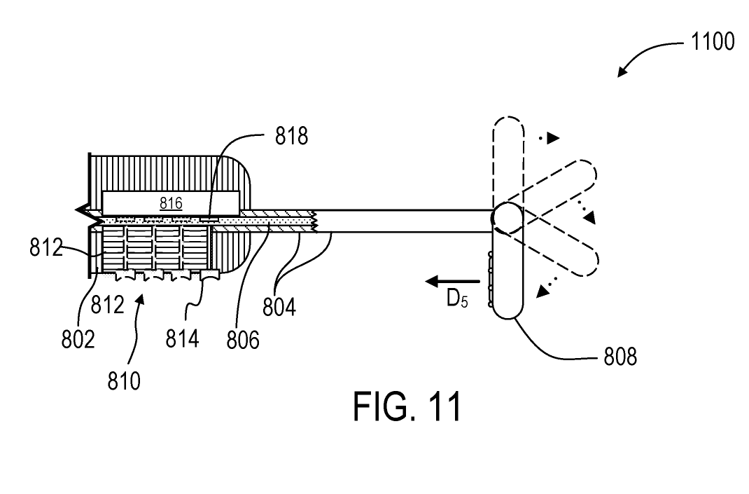

FIG. 11 illustrates an example embodiment of a multi-position treatment device 1100 that includes a tiltable treatment element 1108. The treatment element 1108 can be tilted to allow an adjustable treatment direction D₅ within a plane substantially parallel to the length of the treatment device 1100. The tiltable treatment element 1108 may be achieved in a variety of ways. In one example, the inner shaft 1106 is slidably disposed within the outer shaft 1104, and there is a coupling or other linkage where the outer shaft 1104 meets the treatment element 1108, such that movement of the inner shaft 1106 causes the treatment element 1108 to tilt. For example, the peg 1114 may be a movable button portion that can be slid along the channel 1112, thereby moving the inner shaft 1106 within the outer shaft 1104, thereby causing the treatment element 1108 to tilt and change the treatment direction D₅. Again, there may be a position sensor 1118 and a position indicator 1120 arranged to detect a bend or other configuration of the treatment element 1108. Multiple exemplary positions of the treatment element 1108, pegs 1114, and position indicator 1120 are illustrated in dashed lines.

FIG. 11 further illustrates the fixation element 1110 disposed within the handle 1102. In particular, there is a channel 1112 extending through the handle 1102 and in communication with the inner shaft 1106, such that the channel 1112 allows movement of the peg 1114 when the peg 1114 is connected to the inner shaft 1106. The peg 1114 is formed as a sliding button with a concavity for receiving the clinician's thumb.

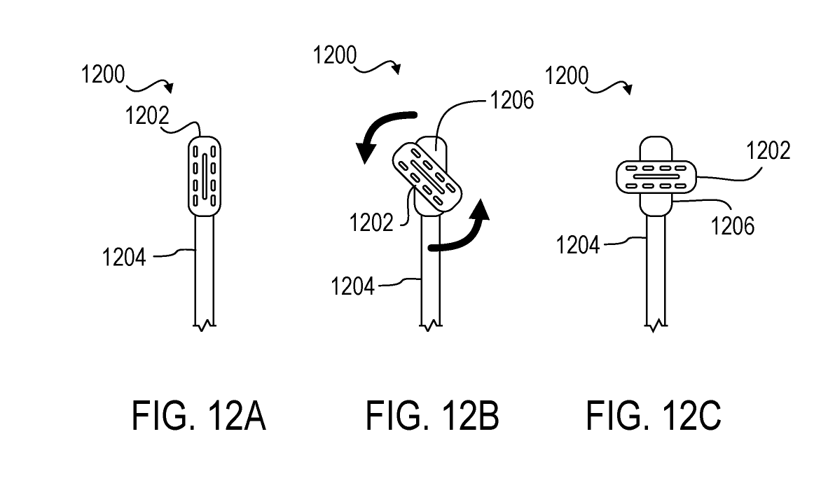

FIGS. 12A-12C illustrate a distal end of an example treatment device 1200 having a rotatable treatment element 1202 that can rotate relative to a shaft 1204, in a plane substantially parallel to the length of the treatment device 1200. Upon actuation of the components, the treatment element 1202 can rotate relative to the shaft 1204. For example, there may be a backing component 1206 that serves as a base on which the treatment number 1202 can rotate. There may be gears or other rotating components within the backing component 1206 that allow for rotation of the treatment element 1202. FIG. 12B illustrates the treatment element 1202 moving in a direction indicated by arrows to reach the position illustrated in FIG. 12C. FIG. 12C illustrates an example arrangement of the treatment device 1200 after rotating the treatment element 1202 relative to the shaft 1204.

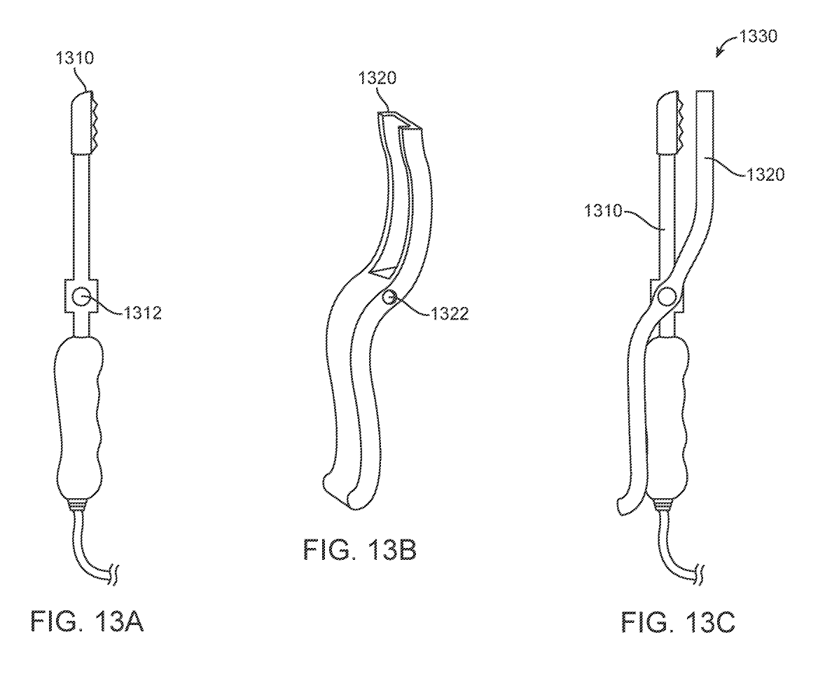

FIGS. 13A-13C illustrate an example of a two-piece, reconfigurable treatment device 1330. Referring to FIG. 13A, a base treatment device 1310 can include an attachment element 1312. FIG. 13B shows a supporting feature 1320 with a complementary attachment element 1322. The base treatment device 1310 can be coupled to the supporting feature 1320 via their respective attachment elements 1312, 1322, to form the combined, two-piece treatment device 1330 of FIG. 13C. The two-piece device can be used in a clamping or tissue-holding procedure, in which tissue is held between the two parts 1310, 1320 of the device 1330.

In the illustrated example, the base treatment device 1310 may include any of the aspects and features described above, for example including a treatment element having bipolar electrodes configured to deliver energy to a treatment site. The supporting feature 1320 may include a clamp portion, an electrode array, an incision forming device, a second treatment device (e.g., to allow for treatment via two nostrils simultaneously), a positioning device, a treatment device configured to provide a different treatment modality than that provided via the base treatment device 1310 (e.g., cryotherapy, chemical, etc.), a sensor array and/or any other such features.

In some examples, one or both of the attachment elements 1312, 1322 or another portion of one or both of the treatment device parts 1310, 1320 may include one or more sensors to determine whether a supporting feature 1320 is attached to the base treatment device 1310 and what kind of supporting feature 1320 is attached. The sensors can provide an output based on the supporting feature 1320 (or lack thereof), and the output can be used to change parameters by which the base treatment device 1310 or the combined treatment device 1330 operates. For example, upon detecting that a clamp supporting feature 1320 is attached, clamp treatment parameters can be automatically selected and used.

Although various embodiments are described herein, the present invention extends beyond the specifically disclosed embodiments to other alternative embodiments and/or uses of the invention and modifications and equivalents thereof. Thus, the scope of the present invention should not be limited by the disclosed embodiments, but should be determined only by a fair reading of the claims that follow.

What is claimed is:

1. A device for treating airway tissue, the device comprising:
a handle;
an outer shaft fixedly attached to the handle;
an inner shaft disposed in the outer shaft; and
a treatment element attached to a distal end of the inner shaft and configured to deliver energy to the airway tissue to modify at least one property of the tissue,
wherein the inner shaft is free to move in at least one direction within the outer shaft to change an orientation of the treatment element from a first configuration to a second configuration.

2. The device of claim 1, wherein the inner shaft is free to translate, relative to the outer shaft, and wherein the first configuration is a retracted configuration and the second configuration is an extended configuration.

3. The device of claim 1, wherein the inner shaft is free to rotate in a plane perpendicular to a length of the inner shaft, relative to the outer shaft, and wherein the treatment element faces in a first direction in the first configuration and a second direction in the second configuration.

4. The device of claim 3, wherein the inner shaft is also free to translate, relative to the outer shaft.

5. The device of claim 1, wherein the treatment element is adjustable relative to the inner shaft, to change an orientation of the treatment element.

6. The device of claim 5, wherein the treatment element is configured to rotate in a plane parallel to a length of the inner shaft.

7. The device of claim 1, further comprising a position sensor for determining whether the treatment element is in the first configuration or the second configuration.

8. The device of claim 7, wherein the device is configured to operate using a first set of parameters when the treatment element is in the first configuration and using a second set of treatment parameters when the treatment element is in the second configuration.

9. The device of claim 1, wherein the outer shaft comprises a lumen and the inner shaft is disposed in the lumen.

10. The device of claim 1, further comprising a fixation mechanism configured to lock the device in the first configuration or the second configuration.

11. The device of claim 1, wherein the treatment element is sized to fit through a nostril of a nose to treat nasal airway tissue.

12. A method for treating an airway, the method comprising:
obtaining an airway treatment device;
actuating a fixation mechanism of the treatment device;
transitioning the device from a first configuration to a second configuration;
positioning a treatment element within the airway proximate an airway tissue to be treated; and
applying energy to the airway tissue with the treatment element, wherein the airway tissue at least partially maintains a modified property after the treatment element is removed and the airway tissue heals.

13. The method of claim 12, wherein actuating the fixation mechanism comprises moving a peg out of a landing.

14. The method of claim 12, wherein transitioning the device from the first configuration to the second configuration comprises moving an inner shaft of the treatment device relative to an outer shaft of the treatment device.

15. The method of claim 14, wherein moving the inner shaft relative to the outer shaft comprises rotating the inner shaft.

16. The method of claim 14, wherein moving the inner shaft relative to the outer shaft comprises translating the inner shaft.

17. The method of claim 12, wherein transitioning the device from the first configuration to the second configuration comprises adjusting a position of the treatment element relative to a shaft of the treatment device.

18. The method of claim 17, wherein adjusting the position of the treatment element comprises rotating the treatment element in a plane parallel to a longitudinal axis of the shaft.

19. The method of claim 12, wherein the airway tissue comprises nasal airway tissue, and wherein positioning the treatment element comprises advancing the treatment element through a nostril.

20. An airway treatment system, comprising:
a treatment device configured to deliver energy to tissue within an airway to modify at least one property of the tissue; and
a control system coupled to the treatment device and configured to:
determine whether the treatment device is in a first configuration or a second configuration; and
modify one or more treatment parameters of the treatment device based on whether the treatment device is in the first configuration or the second configuration.

21. The system of claim 20, wherein the treatment device further comprises a supporting feature removably coupled to an attachment mechanism, wherein the supporting feature is a component selected from the group consisting of a clamp portion, an electrode array, an incision forming device, a second treatment device, a positioning device, and a sensor array.

22. The system of claim 20, wherein the treatment device comprises a sensor configured to determine a configuration of the treatment device, and wherein the control system is configured to determine whether the treatment device is in the first configuration or the second configuration based on an output of the sensor.

23. The system of claim 20, wherein the device comprises a fixation mechanism.

24. The system of claim 20, wherein the device comprises a treatment element comprising multiple selectable treatment portions.

25. The system of claim 24, wherein the first configuration comprises an inner shaft of the plurality of selectable treatment portions being selected, and wherein the second configuration comprises an outer shaft of the plurality of selectable treatment portions being selected.
